(19) United States
(12) Patent Application Publication
(10) Pub. No.: US 2004/0235009 A1
(43) Pub. Date: Nov. 25, 2004

(54) Method for analyzing DNA of sweet potato

(75) Inventors: Maria Berenyi, Eisenstadt (AT); Kornel Burg, Eisenstadt (AT); Simon T. Gichuki, Nairobi (KE); Joseph Schmidt, Eisenstadt (AT)

Correspondence Address: Mark B. Wilson, Fulbright & Jaworski L.L.P., Suite 2400, 600 Congress Avenue, Austin, TX, 78701, US

(73) Assignee: Austria Research Centers GMBH-ARC, Vienna (AT)

(21) Appl. No.: 10/714,820

(22) Filed: Nov. 17, 2003

Related U.S. Application Data
(63) Continuation of application No. PCT/EP02/05216, filed on May 13, 2002.

(30) Foreign Application Priority Data
May 16, 2001 (AT) A 777/2001

(51) Int. Cl. C12Q 1/68

(52) U.S. Cl. 435/6

(57) ABSTRACT

Described is a method for analysing DNA of a sweet potato, characterised in by the following steps:

providing DNA of a sweet potato,

physically breaking said DNA into DNA pieces,

introducing known sequences at at least one of the two ends of each DNA piece,

providing at least two primers, a first primer according to the formula

(Nₓ)ₙAGTCCTAACAN₁N₂N₃ (I)

wherein Nₓ is selected from A, C, G and T; n is 0 to 20; N₁ is G, T, A or not present; N₂ is A, C, G or not present; N₃ is A, C, G or not present; or a complementary sequence thereto; and a second primer being able to anneal to the introduced sequence,

amplifying DNA of the DNA pieces with said primers and

analysing said amplified DNA.

### CROSS-REFERENCE TO RELATED APPLICATIONS

This application is a continuation of International Application No. PCT/EP02/05216 filed 13 May 2002, which claims priority to Austrian Application No. A 777/2001 filed 16 May 2001, the entire disclosures of which are incorporated herein by reference.

### BACKGROUND OF THE INVENTION

The invention relates to a method for analysing DNA of sweet potato. After Columbus introduced the sweet potato to Spain it spread to Africa, India, Asia and Oceania and became an important crop in those parts of the world. It is possible that the spread of the sweet potato outside America was restricted to a limited number of genotypes. Contrary to this supposition a wide variety of phenotypes (genotypes) can be found all over the world, which could be the consequence of the high level of heterozygoticy found in sweet potato. The sweet potato is an out-crossing hexaploid and the variation due to sexual reproduction and somatic mutation can be kept through vegetative propagation.

Several germplasm collections exist throughout the world; the CIP (Lima) has assembled more than 4000 accessions of sweet potato. The maintenance of the large number of varieties is a huge effort, which makes important to quantify the level of diversity of the sweet potato accessions to enable the reduction of the number of the stored samples thus facilitating germplasm conservation.

Several marker systems were developed during the last decades for genotyping that could be applied to the sweet potato such as RAPD (Jarret et al., 1992; Gichuki et al., SSR (Tautz 1988) and AFLP (Zabeau and Vos 1993, Gichuki et al.). The Amplified Fragment Length Polymorphism (AFLP) and Simple Sequence Repeats (SSR) or microsatellites, have recently become popular in fingerprinting and phylogenetic studies. It has also been reported that AFLP assays have better reproducibility across laboratories than RAPDs (Jones et al., 1997), however AFLP sites were shown to be clustered within the genome thus making the construction of linkage maps difficult.

Waugh and his co-workers have developed a new method, called Sequence-Specific Amplified Polymorphism (S-SAP) (Waugh et al., 1997). This method is similar to AFLP but the S-SAP system produces amplified fragments containing long terminal repeat (LTR) sequence of retrotransposon at one end and a flanking adapter sequence ligated to host restriction site at the other displaying individual retrotransposon insertions as bands on a sequencing acrylamide gel (Ellis et al., 1998; Waugh et al., 1997).

Waugh et al. using the original AFLP protocol digest the barley genomic DNA with two restriction endonucleases, a rare (PstI) and frequent (MseI) cutter enzyme and adapt with restriction enzyme digestion site specific adapters. The procedure consist of two consecutive PCRs (polymerase change reactions). In the first one the digested template DNA was pre-amplified to select and bulk restriction fragments of the correct size and configuration using primer homologous (P and M) to the adapter sequences. In the second selective PCR reaction γ-[³³P]ATP labelled Bare-1 like LTR oligonucleotide and P₍₎ or M₍₎ (Pst or Mse specific primers with 1-3 selective nucleotides) selective adapter primers were added. P_{( )} and M_{( )} primers had the same sequence as the P and M primers in the first reaction but included one to three additional selective nucleotides at the 3′end. The touchdown PCR protocol of Vos et al. (1995) was followed exactly.

A considerable advantage of retrotransposon based polymorphic marker system is based on the fact that the Class I retrotransposons transpose via an RNA intermediate, which they convert to DNA by reverse transcription before reinsertion whereas the parental transposon remains fixed in the genome (see review Boeke 1989; Kumar 1996). This means that the inserted transposon does not change its position during the evolution of the genome but every insertion elevates the polymorphism and the size of the genome. However solo LTR sequences, found in different genomes indicating that unequal crossing over and/or intrachromosomal recombination events could delete inserted retrotransposon sequences (Shirasu et al., 2000).

Retrotransposons are present in the genomes of all plants, ranging from single cell algae to angiosperms and gymnosperms. They are usually present in high copy number (from hundreds to millions) and high level of heterogeneity (amino acid similarities between individual fragments could vary from 5-75%) was observed among them (Flavell et al. 1992a Mol Gen). Compared with the Drosophila copia, the fungal Ty1 or even animal retrotransposons, in plants they show a considerable degree of sequence heterogeneity and insertional polymorphism, both within and between species (Flavell 1992; Boeke and Corces 1989). The most studied group of LTR retrotransposons is the Ty1-copia group, named after the best-studied elements in Saccharomyces cerevisiae and Drosophila melanogaster (Boeke and Corces 1989, Grandbastien 1989, Schmidt 1996). The LTR sequences are positioned as direct repeats on both ends of the retrotransposons. Different retrotransposon families have different (non-cross-hybridising) LTR sequences. The 5′ and 3′LTR sequences are identical at the time of the insertion but they can be differing through mutations during the time.

Phylogenetic analyses of the retrotransposon sequences show, with some significant exceptions, that the degree of sequence divergence in Ty1-copia retrotransposon populations between any pair of species is generally proportional to the evolutionary distance between those species (Flavell, 1992b). Several authors have also hypothesised that transposition could increase the genetic variability necessary for organisms to adapt to different environmental conditions and that they may be a major factor in the evolution of higher plants (McClintock, 1984; Schwarz-Sommer and Saedler, 1988; Wendel and Wessler 2000). The chromosomal distribution of the Ty1-copia group of retrotransposons in plants has been studied by in situ hybridisation on metaphase chromosomes and has revealed that these elements are dispersed throughout euchromatin and heterochromatin regions of all chromosomes in plants (Pearce 1996, Schmidt 1996, Heslop-Harrison 1997).

Retrotransposon insertion is not a random event, but is controlled by the element itself and by signals depending on the host organism and on external factors. Stresses and environmental challenges are known to stimulate the expression or the transposition of mobile elements (Mhiri et al., 1997; Grandbastien et al., 1997).

Despite of their abundant distribution the most of the retrotransposon sequences are inactive because of the mutations caused defective structures. The only active retrotransposons known to be mobile are the Tto1, Tnt1 and Tnp2 of tobacco and Tos17 of rice (Grandbastien 1989; Vaucheret 1992; Hirochika 1993; Hirochika 1996; Vernhettes 1997; Okamoto 2000), Bare-1 element of barley and PDR1 of pea (Pearce et al., 1997; Ellis et al., 1998).

The ubiquitous distribution, high copy number and widespread chromosomal dispersion of the retrotransposons in plants provide excellent potential for developing a multiplex, DNA-based marker system.

Several retrotransposon-based marker systems have been reported recently.

Purugganan et al. (1995) restriction site polymorphism analysed on a limited region of the Magellan retrotransposon and was able to discriminate even closely related Zea mays subspecies. Waugh et al. in 1997 published the S-SAP method on barley and found that the level of polymorphism is about 25% higher than that revealed by AFLP. Ellis et al. (1998) amplified sequences between the polypurine track of the PDR1 retrotransposon and the 3′TaqI (frequent cutting enzyme) specific adapter sequence, while Pearce et al. (2000) used the same S-SAP technique with two other pea retrotransposon LTR sequences (Tps12 and Tps19) but generating amplified fragments between the 5′LTR and a flanking adapter (TaqI) sequences. Both primer contained selective nucleotides. Both experiments resulted in a detailed picture of the intra and interspecies relationship within the Pisum genus. Gong-Xiu Yu and RP Wisa combined the AFLP, RAPD and S-SAP markers to make a saturated map of diploid Avena based on a recombinant inbred population. Compared with the results of Waugh on barley they also found, that the S-SAP generated markers were more evenly distributed across the Avena genome.

Although Waugh et al. have postulated that their approach may be used as a general approach to obtain linkage information on a range of other conserved sequences in the barley genome and that said approach could also be applied to any other species, its turned out that this S-SAP approach may not be generally applied to phylogenetic analysis of any plant species not even to plant species being similar to barley. One reason for that is that retrotransposon approach according to Waugh et al. is highly dependent on the specific sequence of retrotransposon chosen and also on the general variety of “transposon” jumping.

### SUMMARY OF THE INVENTION

It is an object of the present invention to provide a method for analysing DNA of sweet potatoes allowing phylogenetic and linkage analysis of sweet potato and to provide means for performing this method.

Therefore, the present invention provides a method for analysing DNA of a sweet potato characterised in by the following steps: providing DNA of a sweet potato, physically breaking said DNA into DNA pieces, introducing known sequences at at least one of the two ends of each DNA piece, providing at least two primers, a first primer according to the formula

(Nₓ)ₙAGTCCTAACAN₁N₂N₃ (I)

wherein Nₓ is selected from A, C, G and T; n is 0 to 20; N₁ is G, T, A or not present; N₂ is A, C, G or not present; N₃ is A, G, C or not present; or a complementary sequence thereto; and a second primer being able to anneal to the introduced sequence,

amplifying DNA of the DNA pieces with said primers and

analysing said amplifying DNA.

Surprisingly it turned out with the present invention that a method similar as the one applied by Waugh et al. may be used for analysing sweet potato DNA and making a phylogenetic and linkage analysis of different sweet potato individuals from genetically different sweet potato races. It turned out that a specific retrotransposon of sweet potato, the Str187 retrotransposon, is extremely suitable for analysing and distinguishing even otherwise very closely related sweet potato individuals and allows a clear and distinct phylogenetic grouping of these individuals. In general with the present method a primer designed to the 5′LTR of the Str187 retrotransposon is used together with a primer which is located 5′ to said 5′LTR sequence on an introduced piece of DNA.

The Str187 LTR primer proved to be the most polymorphic of all sequences tested and the sweet potato individuals analysed were found to have an extreme high variability between the numbers of the inserts. Indeed, the gradual increase of the integration sites indicates that the Str187 retrotransposon was/is in the closest past active.

The method according to the present invention further turned out to be much more reliable and specific than other methods tested for this approach in other plant genomes such as RAPD or AFLP.

It is therefore possible to distinguish closely related potato races genetically and allocate them to specific origins.

There is a number of methods known for physically breaking DNA into pieces. Most prominent are statistical or defined restriction endonuclease digestion or mechanical breaking e.g. by sonication. According to the present invention it is preferred to break the DNA by restriction endonuclease digestion, preferably by digestion with at least a 6 bp cutting enzyme, especially EcoRI.

The first primer to be used within the method according to the present invention efficiently amplifies the 5′LTR of retrotransposon Str187. Therefore, the primer preferably comprises in its (Nₓ)₄-region further residues being complementary to said region. (Nₓ)₄ is therefore preferably e.g. TAAGACTAAG (SEQ ID NO:2) or AGACTAAG or even longer sequences from the 5′LTR.

Since 5′LTR sequences are identical or at least highly similar to 3′LTR sequences, amplification of DNA pieces comprising 3′LTR sequence might have a negative effect on the method according to the present invention. Therefore, the primers are preferably designed in a way that excludes amplification of sequences being 5′ of 3′LTR sequences e.g. by providing G, T or A as N₁ (because the first base 5′ of the 3′LTR is a G). Such primers may also be used in a second round of performing the present invention, e.g. if the multiplicity of the differences is too high without such a limitation.

Preferred first primers are therefore selected from AGACTAAGAGTCCTAACA (SEQ ID NO:3), AGACTAAGAGTCCTAACAG (SEQ ID NO:4), AGACTAAGAGTCCTAACAT (SEQ ID NO:5), AGACTAAGAGTCCTAACAA (SEQ ID NO:6), AGACTAAGAGTCCTAACAGC (SEQ ID NO:7), AGACTAAGAGTCCTAACAGA (SEQ ID NO:8), AGACTAAGAGTCCTAACAGG (SEQ. ID NO:9), AGACTAAGAGTCCTAACATA (SEQ. ID NO:10), AGACTAAGAGTCCTAACATG (SEQ ID NO:11), AGACTAAGAGTCCTAACATC (SEQ ID NO:12), AGACTAAGAGTCCTAACAAA (SEQ ID NO:13), AGACTAAGAGTCCTAACAAG (SEQ ID NO:14), AGACTAAGAGTCCTAACAAC (SEQ ID NO:15), or fragments thereof, said fragments optionally comprising at least 10 bp of the 3′ part of these sequences.

The introduction of known sequences at at least one of the two ends of each DNA piece (preferably of course at the 5′ end) preferably comprises cutting the DNA with a restriction enzyme, optionally making blunt ends (depending also on the restriction enzyme), and linking an adapter to the end. This adapter comprises e.g. a known sequence whereto said second primer is designed to anneal. Instead of making blunt ends, of course the adapter can be constructed by a linker designed to the restriction site.

The analysis of the amplified DNA is preferably carried out by separating the amplified nucleic acid molecules by size e.g. with gel-electrophoresis. Such systems may be provided in a highly automated form and may be performed by roboters.

The power of the method according to the present invention lies in the fact that it may be used for defining the phylogenetic relationship of any two sweet potato individuals having different genotypes. For defining this relationship a method according to the present invention is performed on each of the sweet potato having different genotypes, thereby getting a defined result with respect to their specific amplification (S-SAP analysis). Then these results of the sweet potato having different genotypes may be compared whit each other. Since with the method according to the present invention each sweet potato gives a characteristic “fingerprint” in this analysis, these fingerprints may be compared to each other and their phylogenetic relationship may be defined by the degree of similarity these fingerprints have. An impressive demonstration of the power of this method is given in the example section.

Preferably the comparing step comprises analysing a size separation of the amplified nucleic acids of each sweet potato species, potato race, potato subtypes, etc. It is therefore possible to differentiate between geographical areas and secondary distribution areas of specific sweet potato specimen.

There is a number of methods for comparing these “fingerprints” preferably these comparisons are performed with computer aids. Several computer programmes are available for such analysis e.g. genotyper. Treecon, TFPGA, Arlequin, Genographer, RFLPSCAN etc.

According to another aspect of the present invention also a kit for performing the methods according to the present invention is provided which comprises at least two primers as defined herein (a first primer and a second primer) and a nucleic acid polymerase for amplifying nucleic acid defined by these two primers.

Preferably, a kit according to the present invention further contains a restriction enzyme specific adapter with primer, a ligase enzyme for the adapter ligation, buffers, nucleotides, positive or negative controls and mixtures thereof.

According to another aspect the present invention also relates to a nucleic acid molecule comprising a sequence of the formula II,

(Nₓ)ₒAGTCCTAACA(Nₓ)ₘ (II),

wherein Nₓ is selected from A, C, G and T; m and o are independently from each other 0 to 1000.

Especially, the present invention provides a nucleic acid molecule comprising SEQ ID NO:1, sequences differing in not more than 1 b/bp per 20 b/bp from this sequence, sequences hybridizing under stringent conditions (e.g.6×SSC, 65° C.) to such sequences or complementary sequences to such sequences.

Preferably, the length of II is between 10 and 500, especially between 12 and 286. Preferably it contains the LTR region and optionally the polypurine tag according to FIG. 1 and FIG. 2.

The present invention will be described in more detail by way of the following examples and the drawing figures, yet it is not restricted to these particular embodiments.

### BRIEF DESCRIPTION OF THE DRAWINGS

FIG. 1 shows Ipomoea batatas retrotransposon partial sequence (3′ RNaseH (SEQ ID NO:30), polypurine track and partial LTR region (SEQ ID NO:1)).

FIG. 2 shows Ipomoea batatas retrotransposon sequence and the used LTR primers (Str6 RNaseH=SEQ ID NO:28, (−1) 3′LTR primer=SEQ ID NO:31; Str85 RNaseH=SEQ ID NO:29, (−1bp 3′ LTR primer=SEQ ID NO:32; Str187 RNaseH=SEQ ID NO:30, (+1bp) 3′ LTR primer=SEQ ID NO:33; Str187/0 primer=SEQ ID NO:3; Str187/G primer=SEQ ID NO:4; Str187/GC primer=SEQ ID NO:3; E01 primer=SEQ ID NO:18; E44 primer=SEQ ID NO:35).

FIG. 3 shows a comparison of the banding pattern after S-SAP analysis.

FIG. 4 shows a comparison of the S-SAP and AFLP analysis of nine sweet potato genotypes.

FIG. 5 shows a S-SAP analysis of nine different sweet potato resources.

FIG. 6 shows a regional map of Eastern Africa showing the original collection sites of sweet potato varieties.

FIG. 7 shows a distribution of the plants in four groups with different insertion number; clear columns represent the range of the insertion number in a group while dark columns show the numbers of the varieties in a given group.

FIG. 8 shows a list of adapters and primers used for AFLP pre-amplification and selective PCR (EcoA1 adapter=SEQ ID NO:19; EcoA2 adapter=SEQ ID NO:20; EO1 primer=SEQ ID NO:18; E33 primer=SEQ ID NO:21; E36 primer=SEQ ID NO:22; MseA1 adapter=SEQ ID NO:23; MseA2 adapter=SEQ ID NO:24; MO1 primer=SEQ ID NO:25; M38 primer=SEQ ID NO:26; M40 primer=SEQ ID NO:27).

FIG. 9 shows a phylogenetic analysis of 173 Eastern African varieties by clustering.

FIG. 10 shows a dendrogram based Nei's (1972) genetic distance method=UPGMA modified from neighbor procedure of PHYLIP Version 3.5;

FIG. 11 shows a supposed distribution of the sweet potato in East Africa.

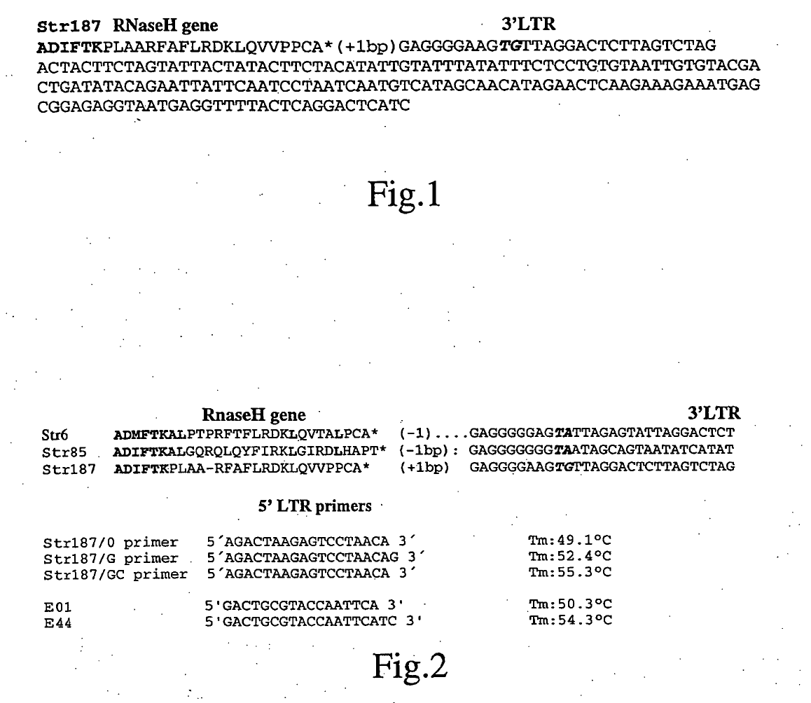

### DETAILED DESCRIPTION

### EXAMPLES

Str187 retrotransposon sequence was found and cloned with a known method (Pearce et al. 1999). After having sequenced the Str187 clones (see SEQ. ID. NO. 1) oligonucleotide primer sequences have been designed capable in different methods to fingerprint and distinguish sweet potato genomes. During the procedure as outlined in the present examples two types of primers are used:

The LTR primer or first primers are designed after the retrotransposon sequence optionally with features preventing amplification of 3′LTR. The other primers or second primers may be any sequence which makes an adapter to the restriction site used, including a primer site. This adapter primer should match the PCR parameters of the first primer. Both primers may be extended on the 3′ end with preferably 1-3 optional nucleotides. In the method according to the present examples the primers are used in PCR reactions with sweet potato DNA templates. The nucleic acid polymerase used in the reactions is a commercially available thermostable DNA polymerase from the thermophilic bacterium Thermus aquaticus (Taq polymerase) or other thermostable polymerases.

The nucleotide triphosphate substrates are employed as described in PCR Protocols, A Guide to Methods and Applications, M. A. Innis et al. 1989 and U.S. Pat. Nos. 4,683,195 and 4,683,204. The substrates can be modified for a variety of experimental purposes in ways known to those skilled in the art.

In the first step (1.) of the present process sweet potato genomic DNA as template DNA is fragmented with sequence specific restriction endonucleases. It is possible to use one, two or even three different restriction endonucleases.

Fragmented genomic DNA is ligated with restriction size compatible adapter sequences with designed adapter specific primer binding sites.

One or more PCR reactions are performed with adapter specific and LTR specific primers. Both primers can be extended with extra nucleotides to reduce the number of the amplified fragments. In the last PCR reaction the LTR primers are labelled so that the LTR-adapter primer amplified PCR product is distinguishable from the adapter-adapter primers.

Such labelling may be performed by any method known in the art. Preferably, labelling by isotopes or non-isotopic methods such as biotinglation, fluorescent dyes or other methods.

PCR-Products may be separated by agarose or acryl amide gel-electrophoresis, manual or automatic, and visualised depending on the labelling of the (LTR) primer.

Similar procedures have been presented from Waugh et al. (1997), Ellis et al. (1998) and Pearce et al. (2000). The electrophoresis of the amplified genomic fragments with the same flanking LTR sequence separates the different length of fragments according to the mobility. Smaller fragments have higher mobility as the longer ones. Different sweet potato samples turned out to have different electrophoresis pattern in consequence with the place and number of the retrotransposon insertions. Automated gel-electrophoresis systems (sequencer equipment, Genotyper programme) can compare more than hundred fragments of different length, but it is of course also possible to evaluate the result with manual methods. Conversion of these electrophoresis patterns to a presence/absence (yes/no) per variety matrix is possible with GENOTYPER or GENOGRAPHER programmes mentioned above or can of course be done manually. A clustering analysis of this matrix is possible using such methods as Unweighted Pair Group Method using Arithmetic Averages (UPGMA) (Sneath and Sokal, 1973) or neighbour joining (Saitou and Nei, 1987) with programmes such as TREECON. Other ordination analysis are also possible such as multidimension scaling (MDS) or principal component analysis (PCO) with programmes such as SPSS, SYSTAT, STATISTIKA or SAS. Further it is possible for the analysis of the geographical origin of the tested sweet potato sample to compare the number of the retrotransposon insertions in the related genotypes. Plants growing on the same area are liable to the same stress effect which could induce among others retrotransposon activation further new insertions.

### Example I

Sweet Potato Resources and DNA Purification

Lyophilised leave samples were used for all the analysis. Sixty seven landraces were obtained from the Kenya Agricultural Research Institute gene banks at the University of Nairobi, field station, Kabete 59 landraces were obtained from the Ugandan National Agricultural Research Institute, Namulongeand. Forty four landraces were obtained from the Tanzania Agricultural Research Institute, Tengeru. Individual pathogen tested clones from Columbia, Peru, Mexico, Brasil and Papua New Guinea were obtained from the International Potato Centre germplasm collection at Kabete, Kenya. From this total sample 9 genotypes from different countries were selected for primer comparisons and for comparison of the S-SAP system with other molecular markers (AFLPs and RAPDs). Details of these genotypes are given in Table 1.

TABLE 1

| | | Other names | | |
|---|---|---|---|---|
| | Variety | and codes | Country of origin | Type of genotype |
| 1 | Mafuta | | Kenya | Landrace |
| 2 | Simama | Kemb 10, | Kenya | Landrace |
| | | CIP 440169 | | |
| 3 | Kyebandula | EAI 56702 | Uganda | Landrace |
| 4 | Wagabolige | CIP 440167 | Uganda | Landrace |
| 5 | Camote | CIP 400014 | Colombia | Landrace |
| | Amarillo | | | |
| 6 | Santo Amaro | CIP 400011 | Brasil | Landrace |
| 7 | No. 221 | CIP 400009 | Mexico | Landrace |
| 8 | Japonese | CIP 420009 | Peru | Landrace |
| | Tresmesino | | | |
| 9 | Naveto | CIP 440131 | Papua New Guinea | Landrace |

Table 1

Names, country of origin and type of genotype of the nine selected varieties used for testing primer combinations and for comparing S-SAP molecular markers with RAPD and AFLP markers

All the KARI (Kenya Agricultural Research Institute) and CIP (International Potato Centre) germplasm was sampled from field collections. For most of the Ugandan and Tanzania germplasm, vine cuttings were sampled from the field collection and planted in pots in a green house. Four weeks later, fresh leaves were sampled for freeze drying. In all cases, 5-7 very young leaves were cut from vigorously growing plants, immediately dipped in liquid Nitrogen. Freeze dried leaves were stored at 4° C. until DNA was isolated. About 20 mg of freeze dried plant material in liquid Nitrogen was ground in a bead mill for 5 minutes. Total DNA was isolated and purified with a ‘Dneasy plant minikit’ (QIAGEN) following the original protocol. After extraction, 4 μl of 10 mg/ml RNase A was added and the sample incubated at 37° C. for one hour. DNA was quantified with a ‘TKO 100’ Mini-fluorimeter (Hoefer scientific instruments) and quality assessed on a 0.8% agarose gel stained with 0.5 μg/μl Ethidium Bromide in a 1× TBE buffer.

PCR Amplification of Ty1-Copia Retrotransposon LTRs

MseI or EcoRI restriction enzyme digested genomic DNA was amplified with degenerate RNaseH gene specific and enzyme cutting site specific flanked PCR primers as written by Pearce et al. 1999. Separation of the biotinilated first PCR products was made on Streptavidin coated magnetic Dynabeads particles.

| | |
|---|---|
| 5′ Biotinilated RNaseH primer: | |
| 5′ MGNACNAARCAYATHGA | (SEQ ID. NO:16) |
| Nested RNaseH primer: | |
| 5′ GCNGAYATNYTNACNAA | (SEQ ID. NO:17 |

N: A, G, T or C
M: A or C
Y: C or T
H: A, C or T

The degenerated RNaseH primers are kindly gift from the laboratory of AJ Flavell (Department of Biochemistry, Univ. Dundee) and were designed by sequence homologies of known retrotransposon origin RNaseH genes. The amplified fragments were cloned into Topo 4 TA cloning vector (TOPO TA Cloning Kit, Clontech K4575-01) and sequenced.

Identification of LTR Sequences of Ty1-Copia Type Transposons of Sweet Potato:

Approximately one hundred clones with variable degree of homology to Ty1-copia RNaseH gene were identified but only three (Str6, Str85, Str187) showed the characteristic RNaseH gene, stop codon, polypurine track and putative 3′LTR sequence elements (FIG. 2). The Str6 and Str187 sequences proved to be homologue with the Ty1-copia retrotransposons. The Str85 clone was not recognised by Blast search as copia type retrotransposon sequence despite the copia homologue primer site in the RNaseH similar sequence and the polypurine track region. The putative inverted repeat region (IR) of the LTR region is different in the three sweet potato sequences, only the Str187 clone contains the characteristic TGTT sequences. Although with lower frequencies other IR sequences occur (Picea abies Tpa8 TAGTT) it is believed already as a mutation. Furthermore, in the putative LTR region of the Str6 clone, after the TATT inverted repeat sequence a 34 bp long direct repeat was recognised which provided another proof for the unusually high mutation rate in the sweet potato retrotransposon population. The starting point of the 3′LTR sequences for the rest of the sequenced clones could not be determined, since they did not contain a recognisable polypurine-track after the RNaseH gene stop codon. In many cases the sequence was interrupted with the MseI restriction cutting site, however using the rare cutting EcoRI enzyme to fragment the genomic DNA longer clones have been got, but the identification of the LTR sequence was further not possible.

The LTR sequence detected in the Str6 and Str187 clones proved to be functional in the S-SAP analysis while Str85 did not produce an amplified polymorphic banding pattern. FIG. 2 shows the list of the LTR and Eco adapter primers tested in S-SAP reactions.

Discussion

PCR Amplification of the Ty1-Copia Retrotransposon LTRs

Sweet potato DNA sequences were isolated with degenerate oligonucleotide primers corresponding to conserved domains of the Ty1-copia retrotransposon RNaseH gene fragment and flanked adapter primers. The amplified clones were cloned as written in Methods. 2-300 random clones have been sequenced but only three clones with recognisable LTR sequences were found. In these three clones the stop codon of the RNaseH genes, the characteristic polypurine tracks and the putative 3′LTR regions could be distinguished.

Every retrotransposon class has a different LTR region, which is homologue in the class, but not between classes. The fact that only two working LTR region were found between more hundred sequenced clones one can suppose, that in the sweet potato the mutation rate of the retrotransposons are very high, and also that only few classes of retrotransposon class exist. Otherwise it has to be considered, that the sweet potato are propagated mainly vegetatively, which means, that a retrotransposon insertion in the vegetative cells has longer “life time” furthermore bigger chance for mutations. It is known that different biotic and abiotic stresses can induce the mobility of the retrotransposon (Mhiri et al., 1997; Grandbastien et al., 1997). However plant genomes have evolved mechanisms to repress uncontrolled retrotransposon expansion, such as DNA methylation (Liu and Wendel 2000) deleterious mutations (Nuzhdin 1999; Heslop-Harrison et al. 1997), unequal crossing over and/or intrachromosomal recombination between LTRs (Shirasu et al. 2000).

The high variability of the Str187 retrotransposon insertion between different sweet potato clones alludes to the mobility of these retrotransposon.

After preliminary experiments the Str187 retrotransposon LTR sequences were used to design S-SAP primers. Increasing the number of the selective nucleotide on the adapter or LTR primers the number of the detected insertions were reduced as expected. However the reduction was much more effective (4-5 times per nucleotide) if more selective nucleotides on the LTR primer were increased the best scoring was achieved with only one nucleotide extension on the LTR primer but it has to be considered that only a 6 bp cutting enzyme was used to fragment the genomic DNA. Waugh et al. fragmented the barley genomic DNA with a 6 and a 4 bp cutting enzyme accordingly to the AFLP procedure, generating more and shorter genomic fragments, but they had to reduce the number of the amplified fragments to a scorable amount with increasing the number of the selective nucleotides. Furthermore, they did not use the selective nucleotide on the LTR primer accordingly they amplified not only the plant specific genomic DNA but possibly the internal retrotransposon sequences too.

S-SAP Method

The procedure from Waugh et al. (1997) was adopted to sweet potato with some modification.

Genomic DNA was digested only with one rear-cutting enzyme (EcoRI) and ligated with specific adapter in one reaction. Two PCR reactions were performed.

The first pre-selective PCR amplification was made with Dynazyme Taq polymerase in 50 μl reactions during 30 cycles on 52° C. annealing temperature. LTR specific primers without any extension and the E01-adapter primer (Table 2) were used.

TABLE 2

| | | |
|---|---|---|
| LTR | | |
| primers | | |
| Str187/0 | 5′ AGACTAAGAGTCCTAACA 3′ | (SEQ ID NO:3) |
| Str187/G | 5′ AGACTAAGAGTCCTAACACG 3′ | (SEQ ID NO:4) |
| Adaptor | | |
| primer | | |
| E01 | 5′ GACTGCGTACCAATTCA 3′ | (SEQ ID NO:18) |

Str187 primers used in S-SAP analysis

First reaction: Str187/0-E01

Second reaction: Str187/G-E01

Second, selective PCR amplification was made with Quiagen Hot Taq DNA polymerase in 25 μl reactions. Touch down from 70° C. (−0.7° C./cycle) to 55° C. than another 20 cycles at 55° C. annealing temperature. With selective nucleotide extended FAM labelled transposon primer (Str187G) was combined with the E01 adapter primer (Table 2). Reactions were loaded on acrylamide gel and separated on ABI 373 automated sequencer.

Adaptation of the S-SAP Method to Sweet Potato

In the original S-SAP protocol (Waugh et al.) the genomic DNA are cut with two enzymes as it is usual in AFLPs, a rare cutter and a frequent cutter (Vos et al.). However adapting the S-SAP technique for sweet potato digesting the genomic DNA with only one rare cutting enzyme instead of two improved the number and length of the polymorphic bands. Further improvement was achieved by pre-amplifying the adapted DNA with the adapter and non-labelled LTR primers. The second specific amplification was carried out with the adapter primer and selective nucleotide extended LTR specific primer. These modifications resulted in a high number of amplified products both polymorphic and monomorphic. In preliminary experiments the three sweet potato LTR primers were tested in S-SAP analysis and the Str187 showed the highest level of polymorphism. The Str6 primer produced a moderate number of polymorphic patterns, but no amplification products were obtained with Str85.

Subsequent experiments were carried out with the Str187 LTR primers.

Nine sweet potato varieties were selected from Africa, South and Central America and Papua New Guinea and tested with the different LTR/adapter primer combinations. Table 3 shows the results of these comparisons.

TABLE 3

| | E44/187GC | | E01/187GC | | E44/187G | | E01/187G | |
|---|---|---|---|---|---|---|---|---|
| Freq. | N | % | N | % | N | % | N | % |
| 1 | 25 | 64 | 32 | 63 | 79 | 46 | 86 | 33 |
| 2 | 3 | 8 | 4 | 8 | 40 | 23 | 51 | 20 |
| 3 | 3 | 8 | 4 | 8 | 24 | 14 | 41 | 16 |
| 4 | 4 | 10 | 3 | 6 | 11 | 6 | 28 | 11 |
| 5 | 3 | 8 | 1 | 2 | 10 | 6 | 19 | 7 |
| 6 | 0 | 0 | 2 | 4 | 4 | 2 | 7 | 3 |
| 7 | 0 | 0 | 4 | 8 | 2 | 1 | 10 | 4 |
| 8 | 0 | 0 | 0 | 0 | 2 | 1 | 12 | 5 |
| 9 | 1 | 2 | 1 | 2 | 1 | 1 | 6 | 2 |
| Ins. | 39 | | 51 | | 173 | | 260 | |

Table 3: Comparison of the different primer combinations in S-SAP analysis of nine sweet potato varieties. Frequencies (Freq.) means that the tested Str187 retrotransposon has insertion into one, two or all of the nine genome. Column N represents the total number of the insertions, which are present in the nine genome one, two or nine times.

Dates are shown also in percentage. In the row insertion (Ins.) are shown the total number of the insertions amplified with the given primer pair.

The E44 adapter primer in combination with the Str187GC or G primers gave 36 and 173 polymorphic bands respectively, representing individual retrotransposon insertions. Reducing the number of the selective nucleotide on the LTR primer significantly elevate the number of the amplified insertions.

The same relation was observed in case of the E01/187GC and E01/187G primers. Reducing the selective nucleotide with one, the number of the amplified insertions elevated from 51 to 261.

The number of the selective nucleotide on the adapter specific primer has only a minor effect on the insertion amplification.

In Table 3 there are presented the frequencies of the insertions amplified from only one, two or even all of the nine plan genomes. It can be seen that the polymorphism is very high; the percentage of the monomorph bands comparing with the total number of the insertions is only 1-2%. However the number of the unique insertions—amplified from only one plant genome—is very high 33-69% of the total insertions.

A phylogenetic analysis of the nine sweet potato varieties with the E01-Str187/G and E44-Str187/G primer combinations are shown in FIG. 5. Both primer combinations distinguish the South American varieties from the African ones. The clones from Mexico and Papua New Guinea were associated to the African types. With the two other primer combinations where the LTR primer is extended with two nucleotides, the South American and African varieties were not differentiated from each other (data not shown).

AFLP Analysis

The AFLP methodology was essentially as described by Vos et al. (1995) but adapted for sweet potato with fluorescent labelling and sequencer running of the gel. Two restriction enzymes, MseI and EcoRI were used to fragment the genomic DNA. The restriction-digested DNA was subsequently ligated to two different synthesised double-stranded oligonucleotides that consists of a short DNA strand and the restriction enzyme recognition site (Table 4). Pre-amplification was done using primers E01 and M01. An annealing temperature of 60° C. was used for 45 cycles. Selective amplification of the PCR products of the pre-amplification was done with primers identical to the pre-amplification primers with an additional 2 selective nucleotides at their 3′ ends (Table 4).

TABLE 4

List of Adapters and primers used for AFLP pre-amplification
and selective PCR

Nucleotide sequence

| | | |
|---|---|---|
| EcoRI-Adapters | | |
| EcoA1 | 5-CTC GTA GAC TGG GTA CC-3 | (SEQ ID NO:19) |
| EcoA2 | 5-AAT TGG TAC GCA GTC-3 | (SEQ ID NO:20) |
| Pre-amplification primer | | |
| E01 | 5-GAC TGC GTA CCA ATT CA-3 | (SEQ ID NO:18) |
| Selective PCR Primers | | |
| E33 | 5-GAC TGC GTA CCA ATT CAA G-3 | (SEQ ID NO:21) |
| E36 | 5-GAC TGC GTA CCA ATT CAC T-3 | (SEQ ID NO:22) |
| Mse1-Adapters | | |
| MseA1 | 5-GAC GAT GAG TCC TGA G-3 | (SEQ ID NO:23) |
| MseA2 | 5-TAC TCA GGA CTC AT-3 | (SEQ ID NO:24) |
| Pre-amplification primer | | |
| M01 | 5-GAT GAG TCC TGA GTA AA-3 | (SEQ ID NO:25) |
| Selective PCR primers | | |
| M38 | 5-GAT GAG TCC TGA GTA AAC T-3 | (SEQ ID NO:26) |
| M40 | 5-GAT GAG TCC TGA GTA AAG C-3 | (SEQ ID NO:27) |

EcoRI selective primers were ABI-FAM fluorescent labelled to prevent occurrence of ‘doublets’ on the gels due to unequal mobility of the two strands of the amplified fragments (Vos et al., 1995). The samples were loaded on a 6% polyacrylamide denaturing gel and run with an ABI Prism 373 sequencer for 10 hours. The gel was scanned and samples extracted using GENESCAN 3.1 programme. The PCR products of selective amplification were visualised. An internal size standard was incorporated into the sample. Visualised peaks indicating position of amplified fragments were analysed with GENOTYPER 2.5 programme to develop a 0/1 (absence/presence) fragment by sample matrix. Peak filter conditions were set to include only peaks with scaled height of at least 30. Selection of categories was done as described above for the S-SAP procedure. Informative products typically fall within 50-450 bp, (Sharbel 1999). Only categories between 50-400 bp were utilised for data analysis.

RAPD Analysis

RAPD amplifications were carried out as described by Williams et al. (1991) with a few modifications as described in Gichuki et al., (2001).

Gel and Data Analysis

Data were analysed with Genotyper 2.5 programme. Peaks, corresponding to an amplified retrotransposon insertion were designated into categories. The tolerance of a category was chosen to be ±0.25-0.5 bp, which means if two amplified fragments show bigger difference than 0.5 or 1 bp, then they were selected as two different categories. Data representing insertions in bp were converted with the Genotyper programme to a presence/absence (1/0) of insertion per variety matrix for use in other phylogenetic programmes such as Treecon.

Comparison of the RAPD, AFLP and S-SAP

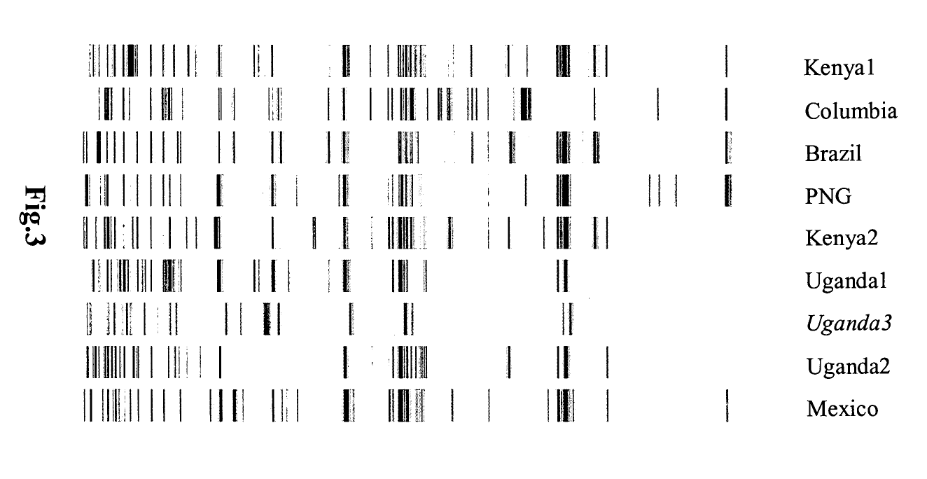

The Ty1-copia transposon based S-SAP analysis is a dominant marker system yielding a multiband pattern. Each individual band of this pattern represents a unique retrotransposon integration site (FIG. 3). The objective was to test whether a genotyping system based on the consecutive integration of retrotransposon elements results in a similar genetic relatedness of accessions compared to those generated using for RAPDs and AFLPs, which are based on the alterations of the DNA sequence. Therefore nine sweet potato genotypes representing different geographic regions already identified by RAPD analysis were analysed by AFLP and S-SAP techniques respectively (Table 1).

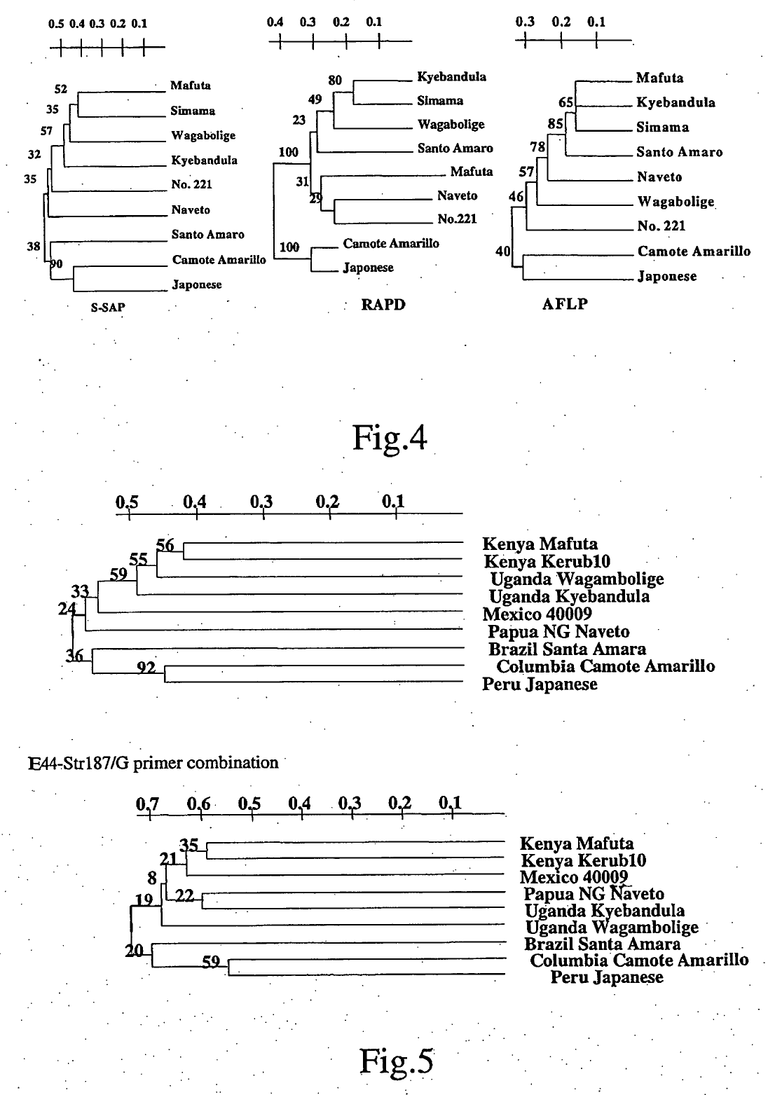

The banding patterns were compared with UPGMA dendograms using Nei, 1979 genetic distance (FIG. 4).

TABLE 5

| | | | Total number | | | |
|---|---|---|---|---|---|---|
| | Number of | Total | of | Number of | Mean number | % |
| | genotypes | number of | amplification | polymorphic | of products | Polymorphic |
| | analysed | assays | products | products | per assay | loci |
| RAPD | 9 | 12 | 74* | 65 | 6.2 | 87.8 |
| AFLP | 9 | 2 | 228 | 179 | 114 | 78.5 |
| S-SAP | 9 | 1 | 260 | 254 | 260 | 97.7 |

Summary of Each Type of Analysis Performed

Total number of amplification products obtained per analysis type, number that were polymorphic, mean number of products per assay (primer or primer product) and overall percentage of polymorphic loci *Only distinct bands which demonstrated polymorphism were scored for RAPDs

Table 5 shows the details of the three analysis methods. The percentage of the polymorphic loci was the highest in S-SAP analysis (97.7%) where 260 insertions were amplified with only one primer pair. In the barley genome, a 25-30% increase in the rate of polymorphism has been observed with retrotransposon-based S-SAP, as compared to standard AFLP (Kumar 1996; Waugh et al. 1997; Gong-Xin Yu and R.P. Wise 2000). In the present case this ration is smaller, 19% comparing with the AFLP method. Although RAPDs showed a high level of polymorphism only distinct banding patterns which showed polymorphism in an earlier study of 74 genotypes were included (Gichuki et al., 2001 in paper). Therefore polymorphism of the RAPD analysis is over-estimated, therefore it is not comparative with the AFLP and S-SAP data (Table 5). The high polymorphism observed in the three methods may be due to the vegetative propagation of the sweet potato.

All the three different genotyping method clearly identified two South American clones Zapallo (Peru)/ and Camote Amarillo (Colombia) as a separate group (see FIG. 4). The four African clones were also identified as another group. The Mexican clone, No.221 and the Papua New Guinea, Naveto, were in all three cases related to the African clones. The Brazilian clone, Santo Amaro, was related to the South American clones in both the S-SAP and the RAPD and with the African clones in the AFLP analysis.

The important factors in choice of a genetic marker includes, development time and cost, capital outlay, amount and quality of DNA required, prior knowledge of DNA sequence, required technical expertise, robustness, informativeness, genome coverage and reproducibility (Vos et al., 1995; Milbourne et al., 1997; Milbourne et al., 1998; Powell et al., 1996). The S-SAP markers require a higher initial cost of development than both RAPDs and AFLPs due to the need to isolate the LTR repeat sequence of the retrotransposon. On the other hand the LTR sequence adaptation costs to specific genomes is comparable to that of AFLPs. The S-SAP was demonstrated to be superior to both RAPD and AFLP in terms of number of amplification products revealed and number of polymorphic loci (Table 5). To select the 12 RAPD random primers more than 100 primers were screened and only about half produced any amplification products. Considering that 12 RAPD assay and 2 AFLP assays were required to achieve approximately the same level of analysis, it is evident that on per assay basis the S-SAP procedure may be the fastest of the three methods for genetic analysis and characterisation of the sweet potato at a comparable cost.

Compared to fluorescent AFLPs it was found that the S-SAP peaks were more distinct. Though both the AFLP and S-SAP markers are dominant, the high multiplex ration of the S-SAPs indicates that they are more informative. AFLP and RAPD markers target random regions of the genome. However some concerns have been expressed by some writers regarding centrometric-clustering of AFLP markers particularly for linkage studies. Most AFLP primers seem to target the AT-rich centromere region of the chromosome. The Ty1-copia retrotransposon is widely distributed throughout the genome (Pearce 1996, Schmidt 1996, Heslop-Harrison 1997). This would mean that the Ty-1 copia LTR S-SAP markers are also widely distributed since they are anchored to the retrotransposon. Reproducibility of a marker system is quite important especially for germplasm characterisation, mapping and where results have to be exchanged between different labs and scientists. The AFLPs have been shown to be more reproducible than RAPDs (Jones et al., 1995). The sequence-specific nature of the S-SAP analysis may improve this reproducibility. Preliminary results indicated a high level of reproducibility using different PCR equipments (data not shown). Considering all these factors it is clear that the Ty-1 copia S-SAP marker system is a powerful method for genetic analysis in sweet potato. The usefulness of retrotransposon S-SAP markers has already been demonstrated in barley (Waugh et al., 1997) and in peas (Elliot et al.).

### Example II

Analysis of the East-African Clones

Hundred seventy-one East-African accessions from Uganda, Tanzania and Kenya were analysed using the E01_{—}187G primer combination in the S-SAP analysis. This primer combination yielded the highest number of polymorphic bands. The PCR amplification and the analysis of the fragments by size were done as described in Materials and Methods.

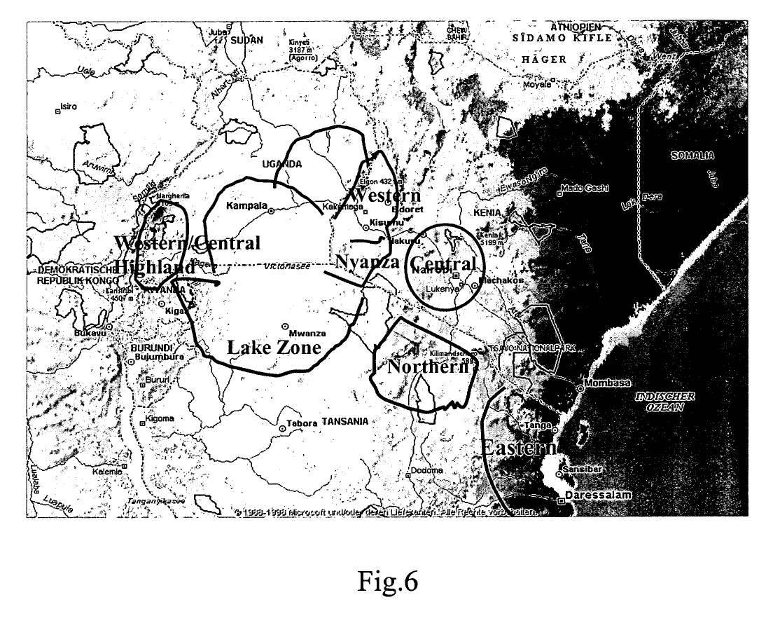

From different areas of East Africa a total of 61 varieties from Kenya, 44 from Tanzania and 61 from Uganda, were selected. Kenyan varieties came from the Central and Western Highlands and the Nyanza region of the Victoria Lake basin. From Tanzania the varieties came from three areas, the East coast, the North-Central Highlands and the Lake zone. Ugandan varieties were grouped into those originating from the North-east Ugandan and the rest originating from Central and Western Uganda The geographical areas of origin are shown in the FIG. 6.

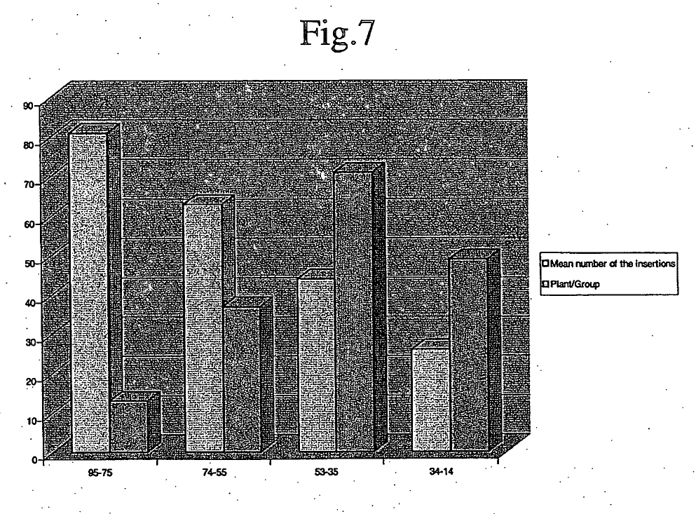

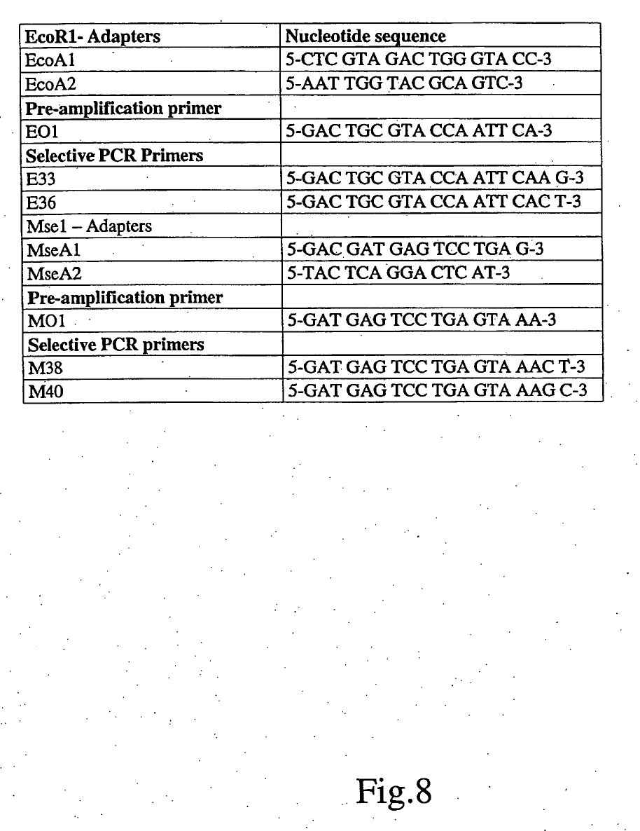

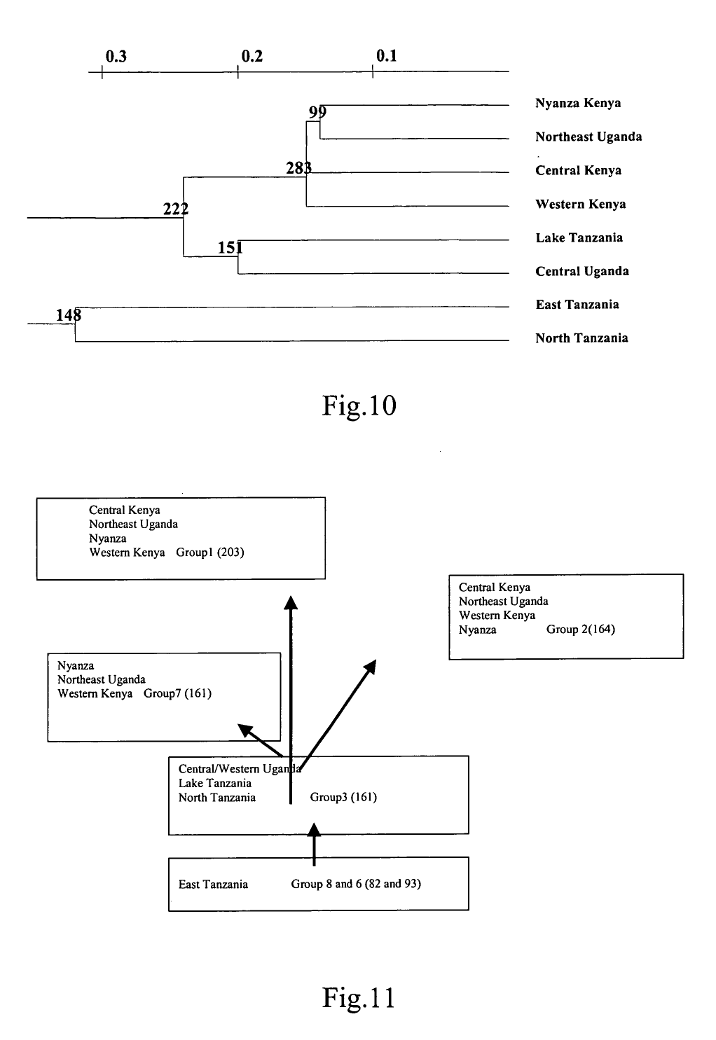

In the S-SAP analysis of all the samples 242 insertions category of the Str187 retrotransposon were found. FIG. 7 present all the varieties in a dendogram based the UPGMA analysis. To simplify the analysis the samples in accordance with the geographical origin or as a member of a given monophyletic group established by Treecon UPGMA analysis were compared. The 172 varieties were first grouped by geographical origin summarised to the given country part then the analysis result was scored and established a phylogenetic tree (see FIG. 10).

The phylogenetic tree shows separation of the East-African sources. East and North Tanzania are separated from the lake part of Tanzania, which is closely related to the Central/West Ugandan samples. These results are corresponding to the geographical position. Interestingly the Northeast Ugandan samples are mapped closer to the Kenyan one than the Central/West Ugandan varieties, but taking considering the geographical localisation it is also feasible. Although the Central Kenyan samples grouped together with the other Kenyan varieties on the phylogenetic tree it is separated from Western and Nyanza part of the country.

The results are correlating with the geographical localisation.

Secondary Distribution of the Retrotransposon Insertions

Comparing the 172 tested varieties with each other by UPGMA cluster analysis ten subgroups have been identified. The subgroups are listed in Table 6.

TABLE 6
Groups based on the phylogenetic analysis

| Gr. 1 | Gr. 2 | Gr. 3 | Gr. 4 | Gr. 5 | Gr. 6 | Gr. 7 | Gr. 8 | Gr. 9 | Gr. 10 |
|---|---|---|---|---|---|---|---|---|---|
| KWA104 | KWA100 | UB101 | KNB22 | KNB2 | TEB158 | KWB1 | KNA28 | KCA113 | TEB148 |
| KWA108 | KCA103 | UB102 | KNB23 | KNB16 | TEB161 | KNB10 | TLB122 | KWA102 | TLB117 |
| KCA117 | KCA105 | UB103 | KNB24 | KWB21 | TEB165 | KNB11 | TLB123 | KCA19 | UNC25 |
| KWA119 | KCA110 | UB104 | KWB18 | KNB25 | TEB166 | KWB12 | TEB125 | UA22 | |
| KNA121 | KNA120 | UB106 | UNC11 | KWB46 | TEB169 | KNB13 | TEB126 | UA4 | |
| KNA131 | KCA129 | UB108 | | KWB3 | TEB159 | KNB15 | TEB127 | KWB20 | |
| KNA134 | KCA36 | UB109 | | KNB47 | TEB152 | KNB26 | TEB128 | UNC4 | |
| KNA135 | TZA52 | UB110 | | KNB14 | TEB131 | KWB27 | TEB129 | | |
| UA32 | UA61 | UB112 | | UNC16 | TZA27 | KNB28 | TEB132 | | |
| KCA38 | KNA86 | UB113 | | UNC2 | KCA7 | KNB29 | TEB138 | | |
| KNA56 | KWA98 | UB114 | | | KWB6 | KNB31 | TEB139 | | |
| KCA77 | KNB19 | TB115 | | | | KNB32 | TEB141 | | |
| KNA79 | UNC21 | TB116 | | | | KNB34 | TEB144 | | |
| KNA80 | UNC24 | TB120 | | | | KNB37 | TEB146 | | |
| KCA85 | UNC29 | TB121 | | | | KNB38 | TEB147 | | |
| KCA94 | UNC31 | TB142 | | | | KNB39 | TEB150 | | |
| KCA99 | UNC35 | KWB54 | | | | KWB4 | TEB151 | | |
| UNC22 | | TNB59 | | | | KNB41 | TEB135 | | |
| UNC26 | | TNB60 | | | | KNB5 | TEB153 | | |
| UNC27 | | TLB61 | | | | KNB51 | TEB155 | | |
| UNC28 | | TNB69 | | | | UNC1 | TEB156 | | |
| UNC30 | | TLB79 | | | | UNC10 | TEB157 | | |
| UNC32 | | UB81 | | | | UNC13 | KWB33 | | |
| UNC33 | | UB83 | | | | UNC14 | TLB75 | | |
| UNC34 | | UB84 | | | | UNC15 | | | |
| UNC36 | | UB86 | | | | UNC18 | | | |
| UNC37 | | UB87 | | | | UNC19 | | | |
| UNC38 | | UB88 | | | | UNC20 | | | |
| | | UB89 | | | | UNC3 | | | |
| | | UB90 | | | | UNC5 | | | |
| | | UB92 | | | | UNC6 | | | |
| | | UB99 | | | | UNC7 | | | |
| | | UNC23 | | | | UNC8 | | | |
| | | | | | | UNC9 | | | |
| Gr. 1 | Gr. 2 | Gr. 3 | Gr. 4 | Gr. 5 | Gr. 6 | Gr. 7 | Gr. 8 | Gr. 9 | Gr. 10 |

This type of analysis shows similar results, but divergence not only between but also in the different country partd to can be observed. The details are shown in Table 7.

TABLE z
Distribution of the varieties in the clustered groups

| | | | No. of possible |
|---|---|---|---|
| | Groups | Percentage | insertion sites |
| Central Kenya | 1 | 43% | 203 |
| | 2 | 36% | 164 |
| Western Kenya | 7 | 25% | 162 |
| | 1 | 18% | 203 |
| Nyanza | 7 | 48% | 162 |
| | 1 | 20% | 203 |
| Central/Western Uganda | 3 | 84% | 161 |
| E/NE-Uganda | 1 | 30% | 203 |
| | 2 | 14% | 164 |
| | 7 | 39% | 162 |
| Tanzania-East | 8 | 66% | 82 |
| | 6 | 28% | 93 |
| Tanzania-Lake | 3 | 60% | 161 |
| | 8 | 30% | |
| Tanzania-North | 3 | 100% | |

The Kenyan varieties are grouped mostly into the Group 1, 2 and 7 together with the Northeast Ugandan ones. For example, 43% of the Central Kenyan clones are in the Group 1 and 33% of them in the Group 2. Similarly the Nyanza clones distributed mainly into the Group 7 but with smaller percent also present in the Group 1 and 2. Western Kenyan samples show the highest diversity, the highest representation is in the Group 7 with 23%, but they can be found also in the Group 1, 2 and 5. The Northeast Ugandan clones show similarity with the Kenyan one, they are mapped into the Group 7, 1 and 2, 39%, 30% and 14% respectively. Much more conserved the Central-Western Ugandan clones, eighty-four percent of them are in the Group 3 together with the three North Tanzanian varieties and 60% of the Lake-Tanzanian samples. Another thirty percent of the Lake Tanzanian varieties are together with the 66% of the East Tanzanian samples in the Group 8. The rest 28% of the East Tanzanian varieties were separated into the Group 6.

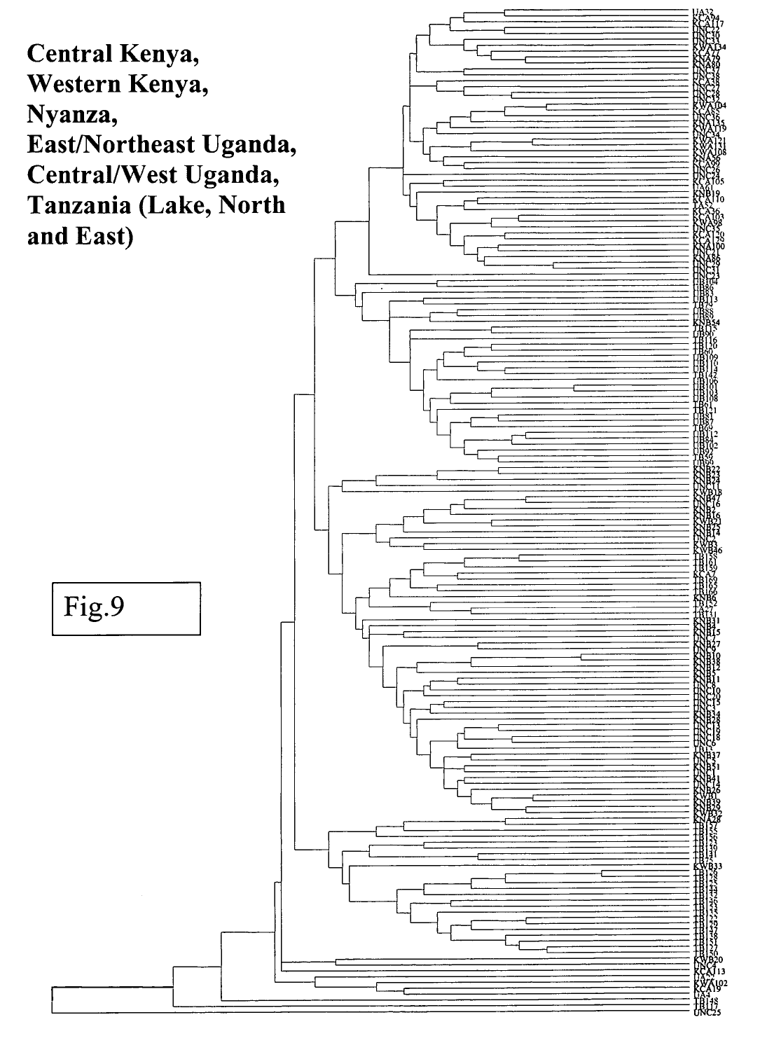

Analysing the number of the insertions in the different groups an increasing number of possible insertion sites from the coast part of Tanzania (East) to Central Kenya has been found. The highest possible insertion number was found in the Group 1 (203). Around 16% of the investigated clones were found in that group, with the highest representation of the Central Kenyan samples (43%). In the group 2, 3, 7 and 9 the number of the possible insertion sites were 164, 161, 162, and around 60-90 possible insertion sites and the most predominant are the East Tanzanian samples in the Group 6 and 8 (see Table 7 and FIG. 9). Table 7 shows only the most characteristic two groups (6, 7), because the others (4, 5 and 10) are too small or too diverse (see also Table 6).

As already mentioned, retrotransposons transpose via an RNA intermediate, which means, that the parental insertion remains fixed in the genome. Therefore every further insertion must have happened later, meaning a recent change in the genome. Continuing this theory the spread of a retrotransposon in the geographical distribution can be followed. In that case one is able to follow the spread of the Str187 retrotransposon in space and time. It is supposed that where the number of the insertion of the given retrotransposon is lower there is the starting point of its spread on a given area. Following this theory and based on the results about the increasing number of the insertions, it is proposed that the sweet potato in East-Africa occurred first in East-Tanzania (insertions 80-90) and spread further to Lake-, North-Tanzania, Central/Western Uganda (ins. 161), East/Northeast Uganda and Kenya (FIG. 11), coming round the Victoria Lake. In Kenya and Northeast Uganda three distribution areas with different insertions rates were found. Varieties from Central, Western and Nyanza area of Kenya are grouped into the Groups 1, 2 or 7 together with the Northeast-Ugandan clones, where the number of retrotransposon insertions is 203, 164 or 162 respectively (see Table 7.). These results could suggest that in Kenya one part of the varieties were exposed to different biotic and abiotic effects, which could induce the retrotransposon expression resulting in new insertions.

Considering the fact, that the sweet potato was introduced into Africa not longer than five hundred years ago and during this time the retrotransposon insertion could increase 2-3 times in the African resources it can be supposed that the Str187 retrotransposon is a still mobile retrotransposon.

### References

Boeke J D, and Corces V G (1989) Transcription and reverse transcription of retrotransposons. Annu Rev Microbiol 43:403-434

Ellis T H N, Poyser S J, Knox M R, Vershinin A V and Ambrose M J (1998) Polymorphism of insertion sites of Ty1-copia class retrotransposons and its use for linkage and diversity analysis in pea. Mol Gen Genet 260:9-19

Flavell A J, Smith D B and Kumar A (1992a) Extreme heterogeneity of Ty1-copia group retrotransposons in plants. Mol Gen Genet 23 1:233-242

Flavell A J, Dunbar E, Anderson R, Pearce S R, Hartley R and Kumar A (1992b) Ty1-copia group retrotransposons are ubiquitous and heterogeneous in higher plants. Nucleic acid Research 20(14): 3639-3644

Gichuki S T, Berenyi M, Zhang D, Hermann M, Schmidt J, Glössl J & Burg K (in preparation) Genetic diversity of Sweet potato [Ipomea batatas (L-) Lam] as assessed with RAPD markers in relationship to geographic sources

Gong-Xiu Yu and Wise R P (2000) An anchored AFLP- and retrotransposon-based map of diploid Avena, Genome 43:736-749

Grandbastien M A, Spielman A, Chaboche M (1989) Tnt1, a mobile retroviral-like transposable element of tobacco isolated by plant cell genetics. Nature 337:376-380

Grandbastien M A, Lucas H, Morel J B, Mhiri C, Vernhettes S and Casacuberta J M (1997) The expression of the tobacco Tnt1 retrotransposon is linked to plant defense responses. Genetica 100:241-252

Heslop-Harrison J S, Brandes A, Taketa S, Schmidt T, Versinin A V, Alkhimova E G, Kamm A, Doudrick R L, Schwarzacher T, Katsiotis A, Kubis S, Kumar A, Pearce S R, Flavell A J and Harrison G E (1997) The chromosomal distributions of Ty1-copia group retrotransposable elements in higher plants and their implications for genome evolution. Genetica 100:197-204

Hirochika H (1993) Activation of tobacco retrotransposons during tissue culture EMBO J 122521-2528

Hirochika H, Sugimoto K, Otsuki J and Kanda M (1996) Retrotransposons rice involved in mutations induced by tissue culture. PNAS USA 93.7783-7788

Jarret R L, Gawel N and Whittemore A (1992) Phylogenetic Relationships of the Sweet potato [Ipomea batatas (L.) Lam] J Amer Soc Hort Sci 117:633-637.

Jones C J, Edwards K J, Castaglione M O, Winfield M O, Sala F, van de Wiel C, Bredemeijer G, Vosman B, Matthes M, Daly A, Brettschneider R, Bettin P, Buiatti M, Maestri E, Malcevschi A, Marmiroli N, Aert R, Volckaert G, Rueda J, Linacero R, Vazquez A and Karp A (1997) Reproducibility testing of RAPD, AFL-P and SSR markers in plants by a network of European laboratories. Molecular breeding 3:381-390.

Kumar A (1996) The adventures of the Ty1-copia group of retrotransposons, TIG 12(2):41-43

Kumar A and Bennetzen J (1999) Plant retrotransposons. Annu Rev Genet 33:479-532

Liu B and Wendel J F (2000) Retrotransposon activation followed by rapid repression in introgressed rice plants. Genome 43:874-880

McClintok B (1984) The significance of responses of the genome to challenge. Science 226:792-801

Mhiri C, Morel J-N, Vernhettes S, Casacuberta J M, Lucas H and Grandbastien M A (1997) The promoter of the tobacco Tn1 retrotransposon is induced by wounding and abiotic stress. Plant Mol Biol 33:257-266

Milbourne D, Meyer R C, Bradshaw J E, Baird E, Bonar N, Provan J, Powell W, Waugh R (1997) Comparison of PCR based marker systems for the analysis of genetic relationship in cultivated potato, Mol. Bred 3:127-136

Milbourne D, Meyer R C, Collins A J, Ramsay L D, Gebhardt C, Waugh R (1998) Isolation and characterisation and mapping of simple sequence repeat loci in potato.

In: Karp A, Isaac P G, Igram D S (eds) Molecular Tools for Screening Biodiversity. Chapman & Hall, London, pp 371-381

Nuzhidin S V (1999) Sure facts, speculations, and open questions about the evolution of transposable element copy number, Genetica 107:129-137

Okamoto H and Hirochika H (2000) Efficient insertion mutagenesis of Arabidopsis by tissue culture-induced activation of the tobacco retrotransposon Tto1i. The Plant Journal 23(2):291-304

Pearce S R, Harrison G, Li D, Heslop-Harrison J. S, Kumar A and Ravell A J (1996) The Ty1-copia group retrotransposons in Vicia species: copy number, sequence heterogeneity and chromosomal localisation. Mol Gen Genet 250:305-315

Pearce S R, Harrison G, Heslop-Harrison J. S, Flavell A J, Kumar A (1997) Characterization and genomic organization of Ty1-copia group retrotransposons in rye (Secale cereale). Genome 40:617-625

Pearce S R, Stuart-Rogers C, Knox M R, Kumar A, Ellis T H N and Flavell A J (1999) Rapid isolation of plant Ty1-copia group retrotransposon LTR sequences for molecular marker studies. The Plant Journal 19(6):711-717

Pearce S R, Knox M, Ellis T H N, Flavell A J and Kumar A (2000) Pea Ty1-copia group retrotransposons: transpositional activity and use as markers to study genetic diversity in Pisum, Mol Gen Genet 263:898-907

Peterson et al., (1993) Adv. Argon. 51:79-123

Powell W, Morgante M, Andre C, Hanafey M, Vogel J, Tingey S, Rafalski A (1996) The utility of RFLP, RAPD, AFLP and SSR (microsatellite) markers for germplasm analysis. Mol Breed. 2:225-238

Purugganan M D and Wessler S R (1995) Transposon signatures: species-specific molecular markers that utilize a class of multiple-copy nuclear DNA. Molecular Ecology 4:265-269

Sharbel F (1999) Amplified Fragment length polymorphisms: A non-random PCR-based technique for multilocus sampling. In: Epplen J T and Lubjuhn T (eds) DMA profiling and DNA fingerprinting. Birkhäuser Verlag, Basel Switzerland pp. 178-194

Saitou, N., Nei, M. (1987) The neighbor-joining method: a new method for reconstructing phylogenetic trees. Mol. Biol. Evol. 4, 406-425.

Schmidt T, Kubis S, Heslop-Harrison J S (1996) Analysis and chromosomal localisation of retrotransposons in sugarbect (Beta vulgaris): LINEs and Ty1-copia-like elements as major components of the genome. Chromosome Res 3:335-345

Shirasu K, Schulman A H, Lahaye T and Schulze-Lefert P (2000) A contiguous 66-kb Barley DANN sequence provides evidence for reversible genome expansion. Genome Research 10:908-915

Sneath, P. H. A., Sokal, R. R. (1973) Numerical Taxonomy. W. H. Freeman, San Francisco.

Studier, J. A., Keppler, K. J. (1988) A note on the neighbor-joining algorithm of Saitou and Nei, Mol. Biol. Evol. 5:729-731.

Swarz-Sommer Z and Saedler H (1988) Transposition and retrotransposition in plants. In: Nelson O (eds) Plant Transposable Elements. Plenum Press, New York, pp 175-187

Tautz D (1988) Hypervariability of simple sequence repeats as a general source for polymorphic DNA markers. Nucleic Acids Res 17:6463-6471

Vaucheret H, Marion-Poll A, Meyer C, Faure J D, Martin E, Caboche M (1992) Interest in and limits to the utilization of reporter genes for the analysis of transcriptional regulation of nitrate reductase. Mol Gen Genet 235:259-268

Vernhettes S, Grandbastien M A and Casacuberta J M (1997) In vivo characterisation of transcriptional regulatory sequences involved in the defence-associated expression of the tobacco retrotransposon Tnt1. Plant Mol Biol 35:673-679

Vos P, Hogers R, Bleeker M, Reijans M, Van de Lee T, Hornes M, Frijters A, Pot J, Peleman J, Kuiper M and Zabeau M (1995) AFLP: a new technique for DNA fingerprinting. Nucleic Acids research 23(21):4407-4414

Waugh R, Mclean K, Flavell A J, Pearce S R, Kumar A, Thomas B B T (1997) Genetic distribution of Bare-1-like retrotransposable elements in the barley genome revealed by sequence-specific amplification polymorphism (S-SAP).

Wendel J F and Wessler S R (2000) Retrotransposon-mediated genome evolution on a local ecological scale. PNAS USA 97(12):6250-6252

Williams J G, Kubelik A R, Livak K J, Raflski J A, Tingey S V (1990) DNA polymorphisms amplified by arbitrary primers are useful as genetic markers. Nucleic Acid Res 18:6531-6535

Zabeau M and Vos P (1993) Selective restriction fragment amplification: a general method for DNA fingerprinting. European Patent Application 924026297

1-12. (Canceled)

13. A method for analyzing DNA of a sweet potato comprising:
providing sweet potato DNA;
breaking the DNA into pieces;
introducing a known sequence at at least one end of the DNA pieces;
providing at least a first primer of formula:
(Nₓ)ₙAGTCCTAACAN₁N₂N₃ (I)
wherein Nₓ is selected from A, C, G and T; n is 0 to 20; N₁ is G, T, A or not present; N₂ is A, C, G or not present; and N₃ is A, C, G or not present; or a complementary sequence thereto; and at least a second primer capable of annealing to the introduced sequence;
amplifying DNA of the DNA pieces with the primers; and
analyzing the amplified DNA.

14. The method of claim 13, wherein breaking the DNA into pieces involves digestion by a restriction endonuclease.

15. The method of claim 14, wherein the restriction endonuclease is a 6 base pair cutting restriction endonuclease.

16. The method of claim 15, wherein the restriction endonuclease is a rare cutting enzyme.

17. The method of claim 13, wherein (Nₓ)₄ residue comprises the sequence AGACTAAG.

18. The method of claim 13, wherein the first primer has a sequence of: AGACTAAGAGTCCTAACA (SEQ ID NO:3), AGACTAAGAGTCCTAACAG (SEQ ID NO:4), AGACTAAGAGTCCTAACAT (SEQ ID NO:5), AGACTAAGAGTCCTAACAA (SEQ ID NO: 6), AGACTAAGAGTCCTAACAGC (SEQ ID NO: 7), AGACTAAGAGTCCTAACAGA (SEQ ID NO:8), AGACTAAGAGTCCTAACAGG (SEQ ID NO:9), AGACTAAGAGTCCTAACATA (SEQ ID NO:10), AGACTAAGAGTCCTAACATG (SEQ ID NO:11), AGACTAAGAGTCCTAACATC (SEQ ID NO:12), AGACTAAGAGTCCTAACAAA (SEQ ID NO:13), AGACTAAGAGTCCTAACAAG (SEQ ID NO:14), AGACTAAGAGTCCTAACAAC (SEQ ID NO:15), or a fragment thereof.

19. The method of claim 13, wherein the first primer is a fragment of a sequence of AGACTAAGAGTCCTAACA (SEQ ID NO:3), AGACTAAGAGTCCTAACAG (SEQ ID NO:4), AGACTAAGAGTCCTAACAT (SEQ ID NO:5), AGACTAAGAGTCCTAACAA (SEQ ID NO:6), AGACTAAGAGTCCTAACAGC (SEQ ID NO:7), AGACTAAGAGTCCTAACAGA (SEQ ID NO:8), AGACTAAGAGTCCTAACAGG (SEQ ID NO:9), AGACTAAGAGTCCTAACATA (SEQ ID NO:10), AGACTAAGAGTCCTAACATG (SEQ ID NO:11), AGACTAAGAGTCCTAACATC (SEQ ID NO:12), AGACTAAGAGTCCTAACAAA (SEQ ID NO:13), AGACTAAGAGTCCTAACAAG (SEQ ID NO:14), AGACTAAGAGTCCTAACAAC (SEQ ID NO:15) and is further defined as comprising at least 10 base pairs of a 3′ region of the sequence.

20. The method of claim 13, wherein introducing known sequences at at least one of end of the DNA pieces comprises cutting the DNA with a restriction enzyme and linking an adapter to the end, the adapter comprising a known sequence.

21. The method of claim 13, wherein analyzing the amplified DNA comprises separating the amplified nucleic acid molecules by size.

22. The method of claim 13, further defined as a method of defining phylogenetic and/or geographical relationships of two or more sweet potatoes having different genotypes.

23. The method of claim 22, comprising analyzing the DNA of a first sweet potato and analyzing the DNA of a second sweet potato and comparing the results.

24. The method of claim 23, wherein comparing the results is further defined as comparing a size separation of amplified nucleic acids from the first sweet potato with a size separation of amplified nucleic acids from the second sweet potato.

25. The method of claim 23, wherein comparing the results comprises using a computer to calculate the phylogenetic distance from a size separation of amplified nucleic acids.

26. A kit comprising a first primer of formula:
(Nₓ)ₙAGTCCTAACAN₁N₂N₃ (I)
wherein Nₓ is selected from A, C, G and T; n is 0 to 20; N₁ is G, T, A or not present;
N₂ is A, C, G or not present; and N₃ is A, C, G or not present; or a complementary sequence thereto;
at least a second primer; and
a nucleic acid polymerase.

27. The kit of claim 26, wherein (Nₓ)₄ residue comprises the sequence AGACTAAG.

28. The kit of claim 26, wherein the first primer has a sequence of: AGACTAAGAGTCCTAACA (SEQ ID NO:3), AGACTAAGAGTCCTAACAG (SEQ ID NO:4), AGACTAAGAGTCCTAACAT (SEQ ID NO:5), AGACTAAGAGTCCTAACAA (SEQ ID NO:6), AGACTAAGAGTCCTAACAGC (SEQ ID NO:7), AGACTAAGAGTCCTAACAGA (SEQ ID NO:8), AGACTAAGAGTCCTAACAGG (SEQ ID NO:9), AGACTAAGAGTCCTAACATA (SEQ ID NO:10), AGACTAAGAGTCCTAACATG (SEQ ID NO:11), AGACTAAGAGTCCTAACATC (SEQ ID NO:12), AGACTAAGAGTCCTAACAAA (SEQ ID NO:13), AGACTAAGAGTCCTAACAAG (SEQ ID NO:14), AGACTAAGAGTCCTAACAAC (SEQ ID NO:15), or a fragment thereof.

29. The kit of claim 26, wherein the first primer is a fragment of a sequence of AGACTAAGAGTCCTAACA (SEQ ID NO:3), AGACTAAGAGTCCTAACAG (SEQ ID NO:4), AGACTAAGAGTCCTAACAT (SEQ ID NO:5), AGACTAAGAGTCCTAACAA (SEQ ID NO:6), AGACTAAGAGTCCTAACAGC (SEQ ID NO:7), AGACTAAGAGTCCTAACAGA (SEQ ID NO:8), AGACTAAGAGTCCTAACAGG (SEQ ID NO:9), AGACTAAGAGTCCTAACATA (SEQ ID NO:10), AGACTAAGAGTCCTAACATG (SEQ ID NO:11), AGACTAAGAGTCCTAACATC (SEQ ID NO:12), AGACTAAGAGTCCTAACAAA (SEQ ID NO:13), AGACTAAGAGTCCTAACAAG (SEQ ID NO:14), AGACTAAGAGTCCTAACAAC (SEQ ID NO:15) and is further defined as comprising at least 10 base pairs of a 3′ region of the sequence.

30. A nucleic acid molecule comprising a sequence of from between 12 and 286 base pairs of SEQ ID NO:1, a sequence differing by not more than 1 base per 20 base pairs from the sequence of SEQ ID NO:1, a sequence that hybridizes under stringent conditions to the sequence of SEQ ID NO:1, or a sequence that is complementary to any of these.

31. The nucleic acid molecule of claim 13, further defined as comprising SEQ ID NO:1.


SEQUENCE LISTING (35 sequences)

SEQ ID NO: 1 - DNA, 195 bp, Artificial Sequence
  Description of Artificial Sequence  Synthetic Primer
gaggggaagtgttaggactcttagtctagactacttctagtattactatacttctacatattgtatttatatttctcctgtgtaattgtgtacgactgatatacagaattattcaatcctaatcaatgtcatagcaacatagaactcaagaaagaaatgagcggagaggtaatgaggttttactcaggactcatc

SEQ ID NO: 2 - DNA, 10 bp, Artificial Sequence
  Description of Artificial Sequence  Synthetic Primer
taagactaag

SEQ ID NO: 3 - DNA, 18 bp, Artificial Sequence
  Description of Artificial Sequence  Synthetic Primer
agactaagagtcctaaca

SEQ ID NO: 4 - DNA, 19 bp, Artificial Sequence
  Description of Artificial Sequence  Synthetic Primer
agactaagagtcctaacag

SEQ ID NO: 5 - DNA, 19 bp, Artificial Sequence
  Description of Artificial Sequence  Synthetic Primer
agactaagagtcctaacat

SEQ ID NO: 6 - DNA, 19 bp, Artificial Sequence
  Description of Artificial Sequence  Synthetic Primer
agactaagagtcctaacaa

SEQ ID NO: 7 - DNA, 20 bp, Artificial Sequence
  Description of Artificial Sequence  Synthetic Primer
agactaagagtcctaacagc

SEQ ID NO: 8 - DNA, 20 bp, Artificial Sequence
  Description of Artificial Sequence  Synthetic Primer
agactaagagtcctaacaga

SEQ ID NO: 9 - DNA, 20 bp, Artificial Sequence
  Description of Artificial Sequence  Synthetic Primer
agactaagagtcctaacagg

SEQ ID NO: 10 - DNA, 20 bp, Artificial Sequence
  Description of Artificial Sequence  Synthetic Primer
agactaagagtcctaacata

SEQ ID NO: 11 - DNA, 20 bp, Artificial Sequence
  Description of Artificial Sequence  Synthetic Primer
agactaagagtcctaacatg

SEQ ID NO: 12 - DNA, 20 bp, Artificial Sequence
  Description of Artificial Sequence  Synthetic Primer
agactaagagtcctaacatc

SEQ ID NO: 13 - DNA, 20 bp, Artificial Sequence
  Description of Artificial Sequence  Synthetic Primer
agactaagagtcctaacaaa

SEQ ID NO: 14 - DNA, 20 bp, Artificial Sequence
  Description of Artificial Sequence  Synthetic Primer
agactaagagtcctaacaag

SEQ ID NO: 15 - DNA, 20 bp, Artificial Sequence
  Description of Artificial Sequence  Synthetic Primer
agactaagagtcctaacaac

SEQ ID NO: 16 - DNA, 17 bp, Artificial Sequence
  Description of Artificial Sequence  Synthetic Primer
mgnacnaarcayathga

SEQ ID NO: 17 - DNA, 17 bp, Artificial Sequence
  Description of Artificial Sequence  Synthetic Primer
gcngayatnytnacnaa

SEQ ID NO: 18 - DNA, 17 bp, Artificial Sequence
  Description of Artificial Sequence  Synthetic Primer
gactgcgtaccaattca

SEQ ID NO: 19 - DNA, 17 bp, Artificial Sequence
  Description of Artificial Sequence  Synthetic Primer
ctcgtagactgggtacc

SEQ ID NO: 20 - DNA, 15 bp, Artificial Sequence
  Description of Artificial Sequence  Synthetic Primer
aattggtacgcagtc

SEQ ID NO: 21 - DNA, 19 bp, Artificial Sequence
  Description of Artificial Sequence  Synthetic Primer
gactgcgtaccaattcaag

SEQ ID NO: 22 - DNA, 19 bp, Artificial Sequence
  Description of Artificial Sequence  Synthetic Primer
gactgcgtaccaattcact

SEQ ID NO: 23 - DNA, 16 bp, Artificial Sequence
  Description of Artificial Sequence  Synthetic Primer
gacgatgagtcctgag

SEQ ID NO: 24 - DNA, 14 bp, Artificial Sequence
  Description of Artificial Sequence  Synthetic Primer
tactcaggactcat

SEQ ID NO: 25 - DNA, 17 bp, Artificial Sequence
  Description of Artificial Sequence  Synthetic Primer
gatgagtcctgagtaaa

SEQ ID NO: 26 - DNA, 19 bp, Artificial Sequence
  Description of Artificial Sequence  Synthetic Primer
gatgagtcctgagtaaact

SEQ ID NO: 27 - DNA, 19 bp, Artificial Sequence
  Description of Artificial Sequence  Synthetic Primer
gatgagtcctgagtaaagc

SEQ ID NO: 28 - protein, 28 aa, Artificial Sequence
  Description of Artificial Sequence  Synthetic Peptide
ADMFTKALPTPRFTFLRDKLQVTALPCA

SEQ ID NO: 29 - protein, 29 aa, Artificial Sequence
  Description of Artificial Sequence  Synthetic Peptide
ADIFTKALGQRQLQYFIRKLGIRDLHAPT

SEQ ID NO: 30 - protein, 26 aa, Artificial Sequence
  Description of Artificial Sequence  Synthetic Primer
ADIFTKPLAARFAFLRDKLQVVPPCA

SEQ ID NO: 31 - DNA, 29 bp, Artificial Sequence
  Description of Artificial Sequence  Synthetic Primer
gagggggagtattagagtattaggactct

SEQ ID NO: 32 - DNA, 29 bp, Artificial Sequence
  Description of Artificial Sequence  Synthetic Primer
gagggggggtaatagcagtaatatcatat

SEQ ID NO: 33 - DNA, 29 bp, Artificial Sequence
  Description of Artificial SequenceRNaseH
gaggggaagtgttaggactcttagtctag

SEQ ID NO: 34 - DNA, 18 bp, Artificial Sequence
  Description of Artificial Sequence  Synthetic Primer
agactaagagtcctaaca

SEQ ID NO: 35 - DNA, 19 bp, Artificial Sequence
  Description of Artificial Sequence  Synthetic Primer
gactgcgtaccaattcatc